(19) United States
(12) Patent Application Publication
(10) Pub. No.: US 2026/0358343 A1
(43) Pub. Date: Nov. 12, 2026

(54) SPECIFIC DETECTION OF HOMODIMERIC HNL

(71) Applicant: DIAGNOSTICS DEVELOPMENT SWEDEN AB, Uppsala (SE)

(72) Inventors: Per Venge, Uppsala (SE); Shengyuan Xu, Uppsala (SE)

(21) Appl. No.: 19/486,540

(22) PCT Filed: May 28, 2024
(86) PCT No.: PCT/EP2024/064616

§ 371 Date: Nov. 21, 2025

(30) Foreign Application Priority Data
Jun. 1, 2023 (SE) 2350677-7

(51) Int. Cl. G01N 33/577; G01N 33/68

(52) U.S. Cl. CPC G01N 33/577; G01N 33/6893; G01N 2800/26

(57) ABSTRACT

Described is a method for detecting a presence or an absence of homodimeric human neutrophil lipocalin (HNL) in a sample. A method, a kit-of-parts and a device for establishing the presence of a bacterial infection or ruling out thereof are also provided. The described method allows for accurate and reliable detection of a bacterial infection and monitoring thereof as well as monitoring efficiency of an antibacterial treatment by specific detection of homodimeric HNL using a specific combination of specific monoclonal antibodies.

### TECHNICAL FIELD

The present invention relates to the methods and means for detecting and monitoring bacterial infections as well as monitoring efficiency of an antibacterial treatment. More specifically, the present invention relates to a method for detecting a presence or an absence of homodimeric human neutrophil lipocalin (HNL) in a sample as well as to a method, a kit-of-parts and a device for establishing a presence or an absence of a bacterial infection.

### BACKGROUND

Bacteria can cause a number of conditions and diseases which are a major burden to society. Among such conditions and diseases are sepsis, gastrointestinal tract infections, urinary tract infections, tuberculosis, pneumonia and gonorrhoea. The treatment of bacterial infections largely relies on administration of antibiotics. Incorrect administration of antibiotics, e. g. administration of antibiotics when treating infections which are not caused by bacteria, may lead to that at least some of the bacteria present in the body become drug resistant. These bacteria may spread into the environment and ultimately become multidrug resistant.

Bacterial antimicrobial resistance (AMR) is currently one of the leading threats to public health, i. e. AMR threatens our ability to treat common infectious diseases. The death rate of patients with infections caused by common drug-resistant bacteria treated in hospitals can be much higher than that of patients with infections caused by the same non-drug-resistant bacteria. For example, patients with infections caused by methicillin-resistant Staphylococcus aureus, a common source of severe infections in the community and in hospitals, are estimated to be 64% more likely to die than patients with infections caused by Staphylococcus aureus that is not methicillin-resistant.

Another serious burden to the society and the health care system is sepsis with about 11 million deaths per year worldwide according to the estimations of WHO. Currently, early diagnosis of sepsis relies mainly on detecting bacterial growth and partly on detecting higher than normal concentrations of biomarkers, such as CRP, procalcitonin and heparin-binding protein. In the majority of cases sepsis is caused by bacterial agents and is treated with antibiotics. To opt for antibiotic treatment, it is important to be able to distinguish sepsis from other conditions, such as systemic inflammatory disease (SIRS). Up to now it has been proven to be difficult with the known in the art methods and means to discriminate between sepsis and SIRS. Sensitivity and specificity for such methods are in the range 70-80% at most.

Therefore, there is an increasing need in the field to reliably detect bacterial infections and to administer antibiotics only to patients who have bacterial infections.

One of the proteins which has been used as a biomarker for detecting a bacterial infection is human neutrophil lipocalin (HNL). HNL is secreted by neutrophils upon their activation and plays a well-established role during an extracellular bacterial infection. The secreted HNL can be detected in bodily fluids.

HNL is a ubiquitous glycoprotein localised in specific granules of neutrophils. HNL can occur as a 25-kD monomer or a 45-kD disulphide-linked homodimer. Furthermore, a part of HNL can be covalently conjugated with gelatinase (matrix metalloproteinase 9) via an intermolecular disulphide bridge forming a 135-kD complex. The amino acid sequence of HNL is shown in SEQ ID NO: 1 corresponding to the protein sequence found in www.uniprot.org/uniprot/P80188, in particular referring to isoform 1.

Sensitivity and specificity for the known in the art serum-based assays which employ measurement of the amount of HNL are in the range 75-94%. The lower figures of the range are obviously not satisfactory for the clinical setting.

EP 0756708 B1 relates to the use of HNL for diagnosing inflammation caused by a bacterial infection. Disclosed immunoassays comprise bringing a sample suspected of containing an abnormal level of HNL in contact with an antibody specific for HNL (anti-HNL antibody). The term anti-HNL antibody means an antibody preparation reacting specifically with HNL, such as in monomeric forms, HNL as included in di-, multi- or heteromeric forms and/or fragments of monomeric HNL exhibiting HNL unique determinants and epitopes.

WO 2016/079219 A1 relates to a binding agent capable of specifically binding to a polypeptide epitope of HNL as well as to the means and methods for detecting a bacterial infection and discriminating between a bacterial infection and a viral infection.

Despite the existing methods, there is still a need for methods and means that would allow for more accurate and reliable detection and monitoring of a bacterial infection as well as monitoring efficiency of an antibacterial treatment. There is also a related need for more accurate and reliable ruling out a bacterial infection.

### SUMMARY

In the light of the above, it is an object of the present invention, as defined in the appended claims, to provide a method for detecting a presence or an absence of homodimeric human neutrophil lipocalin (HNL) in a sample as well as a method, a kit-of-parts and a device for establishing a presence or an absence of a bacterial infection. The invention alleviates at least in part the above-discussed problems and at least partially addresses one or more of the above-mentioned needs. Further details and advantages of the invention will follow from the detailed disclosure below as well as from the dependent claims.

According to a first aspect of the invention an in vitro method is provided for detecting a presence or an absence of homodimeric human neutrophil lipocalin (HNL) in a sample, wherein the method comprises:
i) providing a sample,
ii) providing monoclonal antibodies, a first monoclonal antibody and a second monoclonal antibody, for detecting homodimers of HNL, each of the homodimers consisting of a first monomer and a second monomer, the first monoclonal antibody being configured to bind to the first monomer, the second monoclonal antibody being configured to bind to the second monomer,
iii) bringing the sample into contact with the first monoclonal antibody,
iv) bringing the sample into contact with the second monoclonal antibody,
v) detecting a presence or an absence of homodimeric HNL,
wherein each of the first monoclonal antibody and the second monoclonal antibody comprises a heavy chain variable region and a light chain variable region,
wherein the heavy chain variable region comprises a first set of murine complementarity-determining regions (CDRs), the first set of the murine CDRs comprising:
a first heavy chain CDR (CDRH1), wherein CDRH1 is SEQ ID NO: 6,
a second heavy chain CDR (CDRH2), wherein CDRH2 is SEQ ID NO: 7 and
a third heavy chain CDR (CDRH3), wherein CDRH3 is SEQ ID NO: 8,
wherein the light chain variable region comprises a second set of murine complementarity-determining regions (CDRs), the second set of the murine CDRs comprising:
a first light chain CDR (CDRL1), wherein CDRL1 SEQ ID NO: 9,
a second light chain CDR (CDRL2), wherein CDRL2 SEQ ID NO: 10 and
a third light chain CDR (CDRL3), wherein CDRL3 SEQ ID NO: 11.

According to another aspect of the invention an in vitro method is provided for establishing a presence or an absence of a bacterial infection, wherein the method comprises:
i) providing a sample,
ii) providing monoclonal antibodies, a first monoclonal antibody and a second monoclonal antibody, for detecting homodimers of HNL, each of the homodimers consisting of a first monomer and a second monomer, the first monoclonal antibody being configured to bind to the first monomer, the second monoclonal antibody being configured to bind to the second monomer,
iii) bringing the sample into contact with the first monoclonal antibody,
iv) bringing the sample into contact with the second monoclonal antibody,
v) determining a value corresponding to the amount of homodimeric HNL in the sample,
vi) comparing the value with a control value,
vii) establishing a presence of a bacterial infection when the value is significantly higher than the control value or establishing an absence of a bacterial infection when the value is significantly lower than the control value,
wherein each of the first monoclonal antibody and the second monoclonal antibody comprises a heavy chain variable region and a light chain variable region,
wherein the heavy chain variable region comprises a first set of murine complementarity-determining regions (CDRs), the first set of the murine CDRs comprising:
a first heavy chain CDR (CDRH1), wherein CDRH1 is SEQ ID NO: 6,
a second heavy chain CDR (CDRH2), wherein CDRH2 is SEQ ID NO: 7 and
a third heavy chain CDR (CDRH3), wherein CDRH3 is SEQ ID NO: 8,
wherein the light chain variable region comprises a second set of murine complementarity-determining regions (CDRs), the second set of the murine CDRs comprising:
a first light chain CDR (CDRL1), wherein CDRL1 SEQ ID NO: 9,
a second light chain CDR (CDRL2), wherein CDRL2 SEQ ID NO: 10 and
a third light chain CDR (CDRL3), wherein CDRL3 SEQ ID NO: 11.

The steps of providing a sample and providing monoclonal antibodies may be performed in any order in the methods of the present document. Further, the steps of bringing the sample into contact with the first and the second monoclonal antibody, respectively, may be performed in any order, although it is preferred to bring the sample into contact with the first antibody first.

According to yet another aspect of the invention a kit-of-parts for detecting a presence or an absence of homodimeric HNL in a sample and/or establishing a presence or an absence of a bacterial infection, wherein the kit-of-parts comprises one or more of a monoclonal antibody as described herein, and at least one of instructions for use, a buffer and a device, wherein the device is arranged for analysis of a sample.

According to yet another aspect of the invention a device is provided for establishing a presence or an absence of a bacterial infection, wherein the device comprises:
a first compartment, wherein the first compartment comprises monoclonal antibodies, a first monoclonal antibody and a second monoclonal antibody, for detecting homodimers of HNL, each of the homodimers consisting of a first monomer and a second monomer, the first monoclonal antibody being configured to bind to the first monomer, the second monoclonal antibody being configured to bind to the second monomer,
wherein each of the first monoclonal antibody and the second monoclonal antibody comprises a heavy chain variable region and a light chain variable region,
wherein the heavy chain variable region comprises a first set of murine complementarity-determining regions (CDRs), the first set of the murine CDRs comprising:
a first heavy chain CDR (CDRH1), wherein CDRH1 is SEQ ID NO: 6,
a second heavy chain CDR (CDRH2), wherein CDRH2 is SEQ ID NO: 7 and
a third heavy chain CDR (CDRH3), wherein CDRH3 is SEQ ID NO: 8,
wherein the light chain variable region comprises a second set of murine complementarity-determining regions (CDRs), the second set of the murine CDRs comprising:
a first light chain CDR (CDRL1), wherein CDRL1 SEQ ID NO: 9,
a second light chain CDR (CDRL2), wherein CDRL2 SEQ ID NO: 10 and
a third light chain CDR (CDRL3), wherein CDRL3 SEQ ID NO: 11,
wherein the first compartment is suitable for being brought into contact with a sample, the sample being suspected to contain or not contain homodimers of HNL;
a second compartment, wherein the second compartment comprises a substrate for activating neutrophils present in the sample, wherein the activating occurs prior to the establishing a presence or an absence of a bacterial infection, wherein the second compartment comprises an orifice connecting it with the first compartment;
a first connector, wherein the first connector is arranged to connect the device with another device for removal of a sample from a subject suspected to have at least one of a bacterial infection and a viral infection; and
a second connector, wherein the second connector is arranged to combine the device with a computer and/or an apparatus for analysing interactions between homodimers of HNL, the first monoclonal antibody and the second monoclonal antibody, the analysing permitting determining the amount of homodimeric HNL in the sample.

These and other aspects of the invention are apparent from and elucidated with reference to the embodiments described hereinafter.

### BRIEF DESCRIPTION OF THE DRAWINGS

For exemplifying purposes, the invention will be described in closer detail in the following with reference to embodiments thereof illustrated in the attached drawings.

FIG. 1 graphically illustrates that employing a method according to the present invention (exemplified as enzyme-linked immunosorbent assay (ELISA) with the first monoclonal antibody MAB3 and the second monoclonal antibody MAB3) specific binding and detection of homodimers of HNL can be achieved.

FIG. 2 graphically illustrates that employing a method according to the present invention (exemplified as ELISA with the first monoclonal antibody MAB2 and the second monoclonal antibody MAB2) specific binding to and detection of homodimers of HNL can be achieved.

FIG. 3 graphically illustrates that a method according to the present invention where the same antibody is used as a capture reagent and a detection reagent cannot be performed with any monoclonal antibody specific to HNL. FIG. 3 illustrates that when the antibody MAB5 is used as the first and second monoclonal antibody in a method of the present document, HNL dimers are not detected.

FIG. 4 diagrammatically illustrates levels of homodimers of HNL in plasma samples obtained from patients suffering from acute infections, such as an acute bacterial infection and an acute viral infection. The levels of homodimers of HNL were detected with help of a method according to the present invention employing the first monoclonal antibody MAB3 and the second monoclonal antibody MAB3.

FIG. 5 diagrammatically illustrates levels of homodimers of HNL in plasma samples obtained from patients suffering from acute infections, such as a viral infection, bacterial pneumonia, a mycoplasma infection, tonsillitis, a urinary tract infection (UTI), a gastrointestinal (GI) infection, erysipelas and sepsis. The levels of homodimers of HNL were detected with help of a method according to the present invention employing the first monoclonal antibody MAB3 and the second monoclonal antibody MAB3.

### DETAILED DESCRIPTION OF EMBODIMENTS

In the following detailed description, the technical terms and expressions are defined, and embodiments of the invention are described.

All terms and expressions used in the application text are to be interpreted according to the meaning commonly applied to them in the pertinent prior art unless explicitly defined otherwise herein.

As used in this specification and the appended claims, the singular forms “a”, “an” and “the” include plural referents unless the context clearly dictates otherwise. Thus, e. g. “a first monoclonal antibody” includes one or more first monoclonal antibodies, “a second monoclonal antibody” includes one or more second monoclonal antibodies, “a first monomer” includes one or more first monomers, “a second monomer” includes one or more second monomers.

The terms “first”, “second” and the like are used for distinguishing between similar elements and not necessarily for describing a sequential or chronological order. It is to be understood that each of the terms so used can be substituted with another appropriate term.

The term “comprising” is considered to include the term “consisting of”. If hereinafter a group is defined to comprise at least a certain number of elements, this is meant to also encompass a group which consists of these elements only.

It is to be understood that this invention is not limited to a particular methodology, protocol, proteins, bacteria, vectors, reagents and the like described herein.

The term “antibody” is understood within the scope of the invention to refer to any protein that can be produced by plasma cells in response to an antigen, i. e. a foreign substance that induces an immune response. An antibody, also known as an immunoglobulin, may be exemplified by a peptide comprising natural and/or modified amino acids which permit the peptide to bind to the sequences of the herein described regions of HNL.

The term “monoclonal antibody” is understood within the scope of the invention to refer to an antibody, as that term is defined herein, obtained from a population of substantially homogeneous antibodies, i. e. the individual antibodies comprising the population are identical except for possible naturally occurring mutations or alternative post-translational modifications that may be present in minor numbers. The monoclonal antibodies may be produced by hybridoma cells or with help of a recombinant DNA technique. Non-limiting examples of monoclonal antibodies include murine, rabbit, rat, chicken, chimeric, humanised or human monoclonal antibodies as well as functional fragments thereof, i. e. the fragments exhibiting the desired biological activity. Human monoclonal antibodies may be isolated using transgenic animals that have no endogenous immunoglobulin production and are engineered to contain human immunoglobulin loci.

In accordance with the present invention, the monoclonal antibody or a functional fragment thereof may be linked to e. g. a fluorescent moiety, a radioactive moiety, a chromogenic substrate and the like.

In accordance with the present document, the first monoclonal antibody may alternatively be denoted a “capturing agent”, “capturing reagent”, “capture agent” and the like. Likewise, the second monoclonal antibody may be denoted a “detecting agent”, “detecting reagent”, “detection reagent” and the like.

A typical immunoglobulin (antibody) structural unit is known to comprise a tetramer. Each tetramer is composed of two identical pairs of polypeptide chains, each pair having one “light” chain (about 25 kD) and one “heavy” chain (about 50-70 kD). The N-terminus of each chain defines a variable region of about 100 to 110 or more amino acids primarily responsible for antigen recognition. The term “variable light chain” (VL) refers to a variable region of the light chain. The term “variable heavy chain” (VH) refers to a variable region of the heavy chain. The carboxy-terminal portion of each chain defines a constant region primarily responsible for interactions with other components of the immune system.

The term “complementarity-determining region (CDR)” is understood within the scope of the invention to refer to each of the three hypervariable regions of the variable region of each antibody chain. The variable region is composed of seven amino acid regions, four of which are framework regions and three of which are hypervariable regions. The CDRs are primarily responsible for binding of the antibody to an antigen. The CDRs of each chain are typically referred to as CDR1, CDR2 and CDR3. The CDRs are numbered sequentially starting from the N-terminus and are also typically identified by the chain in which the particular CDR is located. Thus, for example, a VH CDR3 is located in the variable domain of the heavy chain of the antibody, whereas a VL CDR1 is located in the variable domain of the light chain of the antibody. The sequences of the framework regions are relatively conserved within a species.

The term “epitope” is understood within the scope of the invention to refer to a part, or a region, of an antigen which an antibody binds to. An epitope typically includes 5-10 amino acids in a unique spatial conformation. When an antibody recognises a sequence of contiguous amino acids of an antigen (primary structure of the antigen), then the antibody is said to have a linear epitope. When an antibody recognises non-contiguous amino acids juxtaposed by tertiary folding of the antigen, then the antibody is said to have a conformational epitope, also known as a discontinuous epitope. Linear epitopes are typically retained on exposure to denaturing solvents, whereas conformational epitopes are typically lost on treatment with denaturing solvents. Methods of determining spatial conformation of epitopes include x-ray crystallography and 2-dimensional nuclear magnetic resonance.

The term “specific binding”, “specifically binding”, “bind specifically” and the like is understood within the scope of the invention to refer to the ability of the molecule to recognise and bind to a particular target molecule, such as while discriminating against other molecules that are similar in structure and/or composition. The first and second monoclonal antibody of the present document are able to bind specifically to HNL in monomeric and dimeric form.

To mathematically describe the accuracy of a test which reports a presence of a condition or an absence thereof, the terms “sensitivity” and “specificity” are used. If an individual who has the condition is considered “positive” and one who does not have the condition is considered “negative”, then sensitivity is a measure of how well a test can identify true positives and specificity is a measure of how well a test can identify true negatives. Sensitivity is calculated by TP/(TP+FN), wherein TP are “true positives”, FN-“false negatives”. Specificity is calculated by TN/(TN+FP), wherein TN-“true negatives” and FP-“false positives”.

Illustrative embodiments of the invention are described below.

According to a first aspect of the invention a method is provided for detecting a presence or an absence of homodimeric human neutrophil lipocalin (HNL) in a sample, wherein the method comprises:
providing monoclonal antibodies, a first monoclonal antibody and a second monoclonal antibody, for detecting homodimers of HNL, each of the homodimers consisting of a first monomer and a second monomer, the first monoclonal antibody being configured to bind to the first monomer, the second monoclonal antibody being configured to bind to the second monomer,
bringing a sample into contact with the first monoclonal antibody,
bringing the sample into contact with the second monoclonal antibody,
detecting a presence or an absence of homodimeric HNL,
wherein each of the first monoclonal antibody and the second monoclonal antibody comprises a heavy chain variable region and a light chain variable region,
wherein the heavy chain variable region comprises a first set of murine complementarity-determining regions (CDRs), the first set of the murine CDRs comprising:
a first heavy chain CDR (CDRH1), wherein CDRH1 is SEQ ID NO: 6,
a second heavy chain CDR (CDRH2), wherein CDRH2 is SEQ ID NO: 7 and
a third heavy chain CDR (CDRH3), wherein CDRH3 is SEQ ID NO: 8,
wherein the light chain variable region comprises a second set of murine complementarity-determining regions (CDRs), the second set of the murine CDRs comprising:
a first light chain CDR (CDRL1), wherein CDRL1 SEQ ID NO: 9,
a second light chain CDR (CDRL2), wherein CDRL2 SEQ ID NO: 10 and
a third light chain CDR (CDRL3), wherein CDRL3 SEQ ID NO: 11.

The sample may be a bodily fluid. The sample may be derived from a bodily fluid. The bodily fluid may be at least one of blood, plasma, serum, urine, CSF, bone marrow, saliva, sputum, intestinal fluid and another bodily fluid. The sample may also include liquid preparations obtained in various steps during the purification of HNL from its native sources or recombinant production systems. The sample may include at least one of faeces and a liquid preparation thereof. Advantageously, a plasma sample may be used. The sample may be obtained from an animal, such as a mammal, such as a human. The sample may be obtained from a subject suspected to have at least one of a bacterial infection and a viral infection.

The bringing of the sample into contact with the first monoclonal antibody may be preceded by bringing the sample into contact with the second monoclonal antibody.

The sample may be first incubated in the presence of the first monoclonal antibody in a designated space, then the second monoclonal antibody may be added to the designated space. The incubating the sample in the presence of the first monoclonal antibody may be conducted under conditions that allow binding of the first monoclonal antibody to the first monomer of the homodimer of HNL. Similarly, after adding the second monoclonal antibody the time may be allowed for the second monoclonal antibody to bind to the second monomer of the homodimer of HNL, the second monomer being a monomer of the homodimer to which the first monoclonal antibody has not been bound.

Using the combination of the first and second monoclonal antibodies according to the method of the present invention allows not only for specific detection of HNL but for the specific detection of homodimeric HNL. Since it is the homodimeric HNL that is associated with the presence of a bacterial infection, the method according to the present invention allows for more accurate and reliable detection of a presence or an absence of a bacterial infection compared to what can be provided with known in the art methods.

The amino acids of the first monomer to which the first monoclonal antibody binds and the amino acids of the second monomer to which the second monoclonal antibody binds may be the similar. The above CDRs may be configured to bind to an epitope within amino acids 145 to 154 of the first monomer or the second monomer, as defined in SEQ ID NO: 1. The amino acids of the first monomer and those of the second monomer to which the above CDRs may be configured to bind may be determined by X-ray crystallography.

The “binding” may be construed as a binding with an affinity of about 75%, more than about 80%, more than about 81%, 82%, 83%, 84%, 85%, 86%, 87%, 88%, 89%, 90%, 91%, 92%, 93%, 94%, 95%, 96%, 97% 98%, 99% or 100% as assessed e. g. by any one of competitive ELISA, SPR (Biacore) and another suitable method known in the art. The affinity of the binding refers to the strength of the interaction between the two molecules, which is determined by factors such as the shape, charge, and hydrophobicity of the binding sites.

The amino acid sequence of the heavy chain variable region may comprise a sequence as depicted in SEQ ID NO: 5, and/or the amino acid sequence of the light chain variable region may comprise a sequence as depicted in SEQ ID NO: 4.

The amino acid sequence of the heavy chain variable region may comprise a sequence as depicted in SEQ ID NO: 3, and/or the amino acid sequence of the light chain variable region may comprise a sequence as depicted in SEQ ID NO: 2.

The amino acid sequence of at least one of the heavy chain variable region and the light chain variable region of at least one of the first monoclonal antibody and the second monoclonal antibody may be mutated at a single position or multiple positions. Examples of amino acid residues that may be mutated are those that are within a framework region, at least 10% surface exposed and within 4.5 Å of a CDR residue. The amino acid residues to be mutated may be selected by visual inspection of a 3-dimensional structural model of amino acids in proximity to a CDR and/or to one or more selected amino acid residues of a framework region.

At least one amino acid residue of the CDR sequences or the heavy and/or light chain variable region of the above monoclonal antibody (i. e. the first monoclonal antibody and/or the second monoclonal antibody) may be substituted for another amino acid residue, deleted or at least one amino acid residue may be added to an amino acid sequence. The substitutions, additions and/or deletions may be made anywhere in the amino acid sequence, provided the specific binding activity to HNL is maintained. For each variable region/CDR sequence, at least one, two, three, four, five, six or more substitutions, additions and/or deletions may be made, provided the specific binding activity of the above monoclonal antibody to HNL is maintained. In this context the specific binding activity refers to the ability of an antibody to bind specifically to an HNL molecule with a high degree of selectivity and affinity.

At least one of the first monoclonal antibody and the second monoclonal antibody may have a heavy chain comprising the first set of the murine CDRs, wherein the heavy chain comprises a sequence as depicted in SEQ ID NO: 5. At least one of the first monoclonal antibody and the second monoclonal antibody may have a heavy chain comprising the first set of the murine CDRs, wherein the heavy chain comprises a sequence having at least 80%, at least 85%, at least 90%, at least 95%, at least 96%, at least 97%, at least 98%, or at least 99% of identity to SEQ ID NO: 5.

At least one of the first monoclonal antibody and the second monoclonal antibody may have a heavy chain comprising the first set of the murine CDRs, wherein the heavy chain comprises a sequence as depicted in SEQ ID NO: 3. At least one of the first monoclonal antibody and the second monoclonal antibody may have a heavy chain comprising the first set of the murine CDRs, wherein the heavy chain comprises a sequence having at least 80%, at least 85%, at least 90%, at least 95%, at least 96%, at least 97%, at least 98%, or at least 99% of identity to SEQ ID NO: 3.

At least one of the first monoclonal antibody and the second monoclonal antibody may have a light chain comprising the second set of the murine CDRs, wherein the light chain comprises a sequence as depicted in SEQ ID NO: 4. At least one of the first monoclonal antibody and the second monoclonal antibody may have a light chain comprising the second set of the murine CDRs, wherein the light chain comprises a sequence having at least 80%, at least 85%, at least 90%, at least 95%, at least 96%, at least 97%, at least 98%, or at least 99% of identity to SEQ ID NO: 4.

At least one of the first monoclonal antibody and the second monoclonal antibody may have a light chain comprising the second set of the murine CDRs, wherein the light chain comprises a sequence as depicted in SEQ ID NO: 2. At least one of the first monoclonal antibody and the second monoclonal antibody may have a light chain comprising the second set of the murine CDRs, wherein the light chain comprises a sequence having at least 80%, at least 85%, at least 90%, at least 95%, at least 96%, at least 97%, at least 98%, or at least 99% of identity to SEQ ID NO: 2.

Thus, a monoclonal antibody as defined in the present document also encompasses monoclonal antibodies having

The heavy chain of each of the first monoclonal antibody and the second monoclonal antibody may comprise a heavy chain variable region sequence as depicted in SEQ ID NO: 5, and the light chain of each of the first monoclonal antibody and the second monoclonal antibody may comprise a light chain variable region sequence as depicted in SEQ ID NO: 4.

The heavy chain of each of the first monoclonal antibody and the second monoclonal antibody may comprise a heavy chain variable region sequence as depicted in SEQ ID NO: 3, and the light chain of each of the first monoclonal antibody and the second monoclonal antibody may comprise a light chain variable region sequence as depicted in SEQ ID NO: 2.

The amino acid sequence of at least one of the heavy chain constant region and the light chain constant region of the above monoclonal antibody may be mutated at a single position or multiple positions, as described above, provided the specific binding activity of the above monoclonal antibody is maintained.

The amino acid sequence of the first monoclonal antibody may be identical to that of Anti-HNL MAB2 or Anti-HNL MAB3. The amino acid sequence of the second monoclonal antibody may be identical to that of MAB2 or Anti-HNL MAB3.

The amino acid sequence of the first monoclonal antibody may be identical to that of the second monoclonal antibody. The first monoclonal antibody may have the amino acid sequence identical to that of Anti-HNL MAB2, whereas the second monoclonal antibody may have the amino acid sequence identical to that Anti-HNL MAB2. The first monoclonal antibody may have the amino acid sequence identical to that of Anti-HNL MAB3, whereas the second monoclonal antibody may have the amino acid sequence identical to that of Anti-HNL MAB3.

The amino acid sequences of the light chain variable region of Anti-HNL MAB2 is the following:

DIVLTQSTSSLSVSLGDRVTINCRASQDISNYLNWYQEKPDGTVKLLIYF
TSRLHSGVPSRFSGSGSGTDYSLTITNLEQEDIATYFCQQGNTLPWTFGG
GTKLEIKRADAAPTV (see also WO 2016/079219 A1).

The above amino acid sequence is the same as that depicted as SEQ ID NO: 2.
The amino acid sequence of CDRL1 of Anti-HNL MAB2 is the following: RASQDISNYLN.
The amino acid sequence of CDRL2 of Anti-HNL MAB2 is the following: FTSRLHS.
The amino acid sequence of CDRL3 of Anti-HNL MAB2 is the following: QQGNTLPWT.
The amino acid sequences of CDRL1, CDRL2, CDRL3 of Anti-HNL MAB2 are the same as those depicted as SEQ ID NO: 9, SEQ ID NO: 10 and SEQ ID NO: 11, respectively.

The amino acid sequence of the heavy chain variable region of Anti-HNL MAB2 is the following:

EVQLEESGPGLVAPSQSLSITCTISGFSLTSYGIHWLRQPPGKDLEWLVV
IWGDGSTTSNSALKSRLSISKDNSKSQVFFKMSGLQTDDTAIYYCARHRY
SDYHAMDYWGPGTSVTVS (see also WO 2016/079219 A1).

The above amino acid sequence is the same as that depicted as SEQ ID NO: 3.
The amino acid sequence of CDRH1 of Anti-HNL MAB2 is the following: GFSLTSYGIH.

The amino acid sequence of CDRH2 of Anti-HNL MAB2 is the following:

VIWGDGSTTSNSALKS.

The amino acid sequence of CDRH3 of Anti-HNL MAB2 is the following:

HRYSDYHAMDY.

The amino acid sequences of CDRH1, CDRH2, CDRH3 of Anti-HNL MAB2 are the same as those depicted as SEQ ID NO: 6, SEQ ID NO: 7 and SEQ ID NO: 8, respectively.

The amino acid sequence of the light chain variable region of Anti-HNL MAB3 is the following:

DIVLTQTTSSLSVSLGDRVTINCRASQDISNYLNWYQEKPDGTVKLLIYF
TSRLHSGVPSRFSGSGSGTDYSLTITNLEQEDIATYFCQQGNTLPWTFGG
GTKLEIKRADAAPTV (see also WO 2016/079219 A1).

The above amino acid sequence is the same as that depicted as SEQ ID NO: 4.
The amino acid sequence of CDRL1 of Anti-HNL MAB3 is the following: RASQDISNYLN.
The amino acid sequence of CDRL2 of Anti-HNL MAB3 is the following: FTSRLHS.
The amino acid sequence of CDRL3 of Anti-HNL MAB3 is the following: QQGNTLPWT.

The amino acid sequences of CDRL1, CDRL2, CDRL3 of Anti-HNL MAB3 are the same as those depicted as SEQ ID NO: 9, SEQ ID NO: 10 and SEQ ID NO: 11, respectively.

The amino acid sequence of the heavy chain variable region of Anti-HNL MAB3 is the following (see also WO 2016/079219 A1):

EVKLQESGPGLVAPSQSLSITCTISGFSLTSYGIHWLRQPPGKDLEWLVV
IWGDGSTTSNSALKSRLSISKDNSKSQVFFKMSGLQTDDTAIYYCARHRY
SDYHAMDYWGPGTSVTVSS.

The above amino acid sequence is the same as that depicted as SEQ ID NO: 5.
The amino acid sequence of CDRH1 of Anti-HNL MAB3 is the following: GFSLTSYGIH.
The amino acid sequence of CDRH2 of Anti-HNL MAB3 is the following:

VIWGDGSTTSNSALKS.

The amino acid sequence of CDRH3 of Anti-HNL MAB3 is the following:

HRYSDYHAMDY.

The amino acid sequences of CDRH1, CDRH2, CDRH3 of Anti-HNL MAB3 are the same as those depicted as SEQ ID NO: 6, SEQ ID NO: 7 and SEQ ID NO: 8, respectively.

At least one of the first monoclonal antibody and the second monoclonal antibody may be a humanised antibody.

The first monoclonal antibody may be immobilised on a solid support and thereby being configured to bind to the first monomer (of a homodimer of HNL), while the second monoclonal antibody may be in solution and thereby being configured to bind to the second monomer (of the homodimer of HNL), i. e. the monomer that has not been bound by the first monoclonal antibody immobilised on the solid support. This way the first monoclonal antibody may function as a capture reagent, whereas the second monoclonal antibody may function as a detection reagent. A person skilled in the art would appreciate that alternatively the first monoclonal antibody may be configured to bind to the first monomer in a way that is different from that described above. Similarly, a person skilled in the art would appreciate that alternatively the second monoclonal antibody may be configured in bind to the second monomer in a way that is different from that described above.

The above method may further comprise determining the amount of homodimeric HNL in the sample. The amount of homodimeric HNL in the sample may be determined by measuring the amount of the immunocomplexes formed between the above monoclonal antibodies and homodimeric HNL present in the sample. Detection of the immunocomplexes formed may be accomplished by a variety of known techniques, such as radioimmunoassays (RIA) and ELISA, more specifically the double monoclonal antibody sandwich immunoassay technique, as well as immunoradiometric assays, gel diffusion precipitation reactions, immunodiffusion assays, in situ immunoassays (using e. g. colloidal gold, enzyme or radioisotope labels), Western blots, precipitation reactions, agglutination assays (e. g. gel agglutination assays and hemagglutination assays), immunofluorescence assays, protein A assays and immunoelectrophoresis assays.

The above monoclonal antibody may be either labelled or unlabelled. If the antibody is labelled, the reporter group may be any suitable reporter group known in the art, including radioisotope, fluorescent group (e. g. fluorescein or rhodamine), luminescent group, enzyme, biotin and dye particles. Labels that can be indirectly detected by their production of a detectable reaction product include various enzymes, such as alkaline phosphatase, horseradish peroxidase, β-galactosidase, xanthine oxidase, saccharide oxidase, e. g. glucose oxidase, and luciferases. The enzyme can cleave an appropriate substrate to form a coloured reaction product or a fluorescent reaction product. Alternatively, the monoclonal antibody may be detected by a third antibody being able to bind to the first and/or second antibody, the third antibody in turn being labelled as described herein. Typically, in case a third antibody is used for detection, it is configured to bind to the monoclonal antibody used as the detecting agent (see elsewhere herein). Typically, only the one of the first and second antibodies is labelled.

The unlabelled monoclonal antibody may be immobilised on a solid support for use as a “capture agent” (or “capture reagent”) that captures the homodimeric HNL contained within a sample. The solid support may be any material (known to a person skilled in the art) to which the antibody may be attachable. For example, the solid support may be a test well in a microtiter plate or a nitrocellulose or another suitable membrane. Alternatively, the support may be a tube, a bead, a particle or a disc. The support may be of glass, fiberglass, latex or a plastic material, such as polyethylene, polypropylene, polystyrene, polyvinylchloride, or a porous matrix. Other materials that may be used include agarose, dextran, polyacrylamide, nylon, Sephadex, cellulose and polysaccharide. The support may also be a magnetic particle or a fibre optic sensor. In the context of the present invention, the term “immobilisation” refers to both non-covalent association, such as adsorption, and a covalent attachment (which may be a direct linkage between the antibody and a functional group on the support or may be a linkage by way of a cross-linking agent). Immobilisation by adsorption to a well in a microtiter plate or to a membrane is contemplated. In such cases, adsorption may be achieved by contacting the above monoclonal antibody, in a suitable buffer, with the solid support for a suitable amount of time. The suitable duration of the contact depends on the temperature but is typically between about 1 hour and about 1 day. In general, contacting a well of a plastic microtiter plate (including polystyrene or polyvinylchloride) with an amount of the anti-HNL antibody ranging from about 10 ng to about 10 μg, from about 100 ng to about 1 μg, is sufficient to immobilise an adequate amount of the monoclonal antibody. Following immobilisation, the remaining protein binding sites on the support may be blocked. Any suitable blocking agent known to a person skilled in the art, including bovine serum albumin, Tween™ 20 (Sigma Chemical Co., St. Louis, Mo.), heat-inactivated normal goat serum (NGS) or BLOTTO (buffered solution of non-fat dry milk, which also contains a preservative, salts and an antifoaming agent) can be used. Then, the support may be incubated with the sample, i. e. a sample suspected to contain homodimers of HNL. The sample can be applied neat or advantageously can be diluted e. g. in a buffered solution which contains a small amount (0.1%-5.0% by weight) of protein, such as BSA, NGS or BLOTTO.

In accordance with the present document, the first monoclonal antibody may be used as a capture reagent and is immobilised on a surface, while the second monoclonal antibody is used as a detection reagent and is labelled with a label or alternatively detected using a third antibody, which is labelled.

An appropriate duration of contact between the sample and the monoclonal antibody (i. e. incubation time) is a duration of time that may be sufficient to detect the presence of an immunocomplex formed between the monoclonal antibody and homodimeric HNL present in the sample, the monoclonal antibody being immunospecific to homodimers of HNL. The incubation time may be sufficient to achieve a level of binding that is at least about 95% of that achieved at the equilibrium between the bound monoclonal antibody and the unbound monoclonal antibody. A person skilled in the art would appreciate that the time necessary to achieve the equilibrium may be readily determined by monitoring the level of binding that occurs over a period of time. At room temperature an incubation time of about 10 to 30 minutes may be generally sufficient. Thus, after bringing the sample into contact with the first and the second monoclonal antibody, respectively, the sample is incubated in the presence of the respective antibody.

Unbound constituents of the sample may be removed by washing with an appropriate buffer, such as PBS containing 0.1% Tween™ 20. Thus, if a solid support is used for immobilising the first monoclonal antibody, the solid support may be washed after being brought into contact with the sample. Then, a “detection reagent” (i. e. the second monoclonal antibody that binds to the second monomer of a homodimer of HNL) may be added to a space containing an immunocomplex formed by the “capture reagent” (i.e. the first monoclonal antibody) and the homodimeric HNL contained within the sample. The detection reagent may be directly labelled and comprise at least a first detectable label or a “reporter” molecule. Alternatively, the detection reagent (i.e. the second monoclonal antibody) may be an unlabelled anti-HNL antibody. This unlabelled second monoclonal antibody may then be detected by its binding to a third, labelled antibody or a labelled reagent. For example, if the second monoclonal antibody is a murine immunoglobulin, the third antibody may be a labelled anti-murine immunoglobulin antibody. Similarly, if the second monoclonal antibody is a rabbit immunoglobulin, the third antibody may be a labelled anti-rabbit immunoglobulin antibody.

The detection reagent (i.e. the second monoclonal antibody) may be incubated with the immunocomplex formed by the “capture reagent” (i.e. the first monoclonal antibody) and the homodimeric HNL contained within the sample for a suitable amount of time sufficient to detect the bound detection reagent. An appropriate incubation time may be determined by monitoring the level of binding that occurs over a period of time. The detection reagent that remains unbound may be removed, and the amount of immunocomplexes formed between homodimeric HNL, the capture reagent and the detection reagent may then be measured using a suitable assay and a suitable analytical instrument. A person skilled in the art would be able to choose such assay and such analytical instrument. To indicate the presence of a disorder associated with homodimeric HNL, the value corresponding to the amount of immunocomplexes formed between homodimeric HNL, the capture reagent and the detection reagent shall be at least two-fold greater than the control value. The control value may be obtained by measuring the same for a sample obtained from an individual with a normal amount of homodimeric HNL.

Alternatively, the sample and the detection reagent may be contacted simultaneously with the capture agent, rather than sequentially added. In yet another alternative, the sample and the detection reagent may be pre-incubated together in a designated space before adding the capture reagent to the designated space.

The determining of the amount of homodimeric HNL in the sample can be used in a variety of diagnostic, prognostic and monitoring methods, including methods of diagnosing a homodimeric HNL-related disorder, methods of differentiating an inflammatory disease from a non-inflammatory disease and methods of monitoring efficiency of an antibacterial treatment, e. g. for the purpose of tailoring further administration of the antibacterial treatment.

According to a second aspect of the invention a method is provided for establishing a presence or an absence of a bacterial infection, wherein the method comprises:
providing monoclonal antibodies, a first monoclonal antibody and a second monoclonal antibody, for detecting homodimers of HNL, each of the homodimers consisting of a first monomer and a second monomer, the first monoclonal antibody being configured to bind to the first monomer, the second monoclonal antibody being configured to bind to the second monomer,
bringing the sample into contact with the first monoclonal antibody,
bringing the sample into contact with the second monoclonal antibody determining a value corresponding to the amount of homodimeric HNL in the sample,
comparing the value with a control value,
establishing a presence of a bacterial infection when the value is significantly higher than the control value or establishing an absence of a bacterial infection when the value is significantly lower than the control value,
wherein each of the first monoclonal antibody and the second monoclonal antibody comprises a heavy chain variable region and a light chain variable region,
wherein the heavy chain variable region comprises a first set of murine complementarity-determining regions (CDRs), the first set of the murine CDRs comprising:
a first heavy chain CDR (CDRH1), wherein CDRH1 is SEQ ID NO: 6,
a second heavy chain CDR (CDRH2), wherein CDRH2 is SEQ ID NO: 7 and
a third heavy chain CDR (CDRH3), wherein CDRH3 is SEQ ID NO: 8,
wherein the light chain variable region comprises a second set of murine complementarity-determining regions (CDRs), the second set of the murine CDRs comprising:
a first light chain CDR (CDRL1), wherein CDRL1 SEQ ID NO: 9,
a second light chain CDR (CDRL2), wherein CDRL2 SEQ ID NO: 10 and
a third light chain CDR (CDRL3), wherein CDRL3 SEQ ID NO: 11.

More precisely, a method is provided for establishing a presence or an absence of inflammation caused by and/or associated with a bacterial infection. Details regarding the first and second monoclonal antibodies and the bringing into contact and incubation of the sample and the monoclonal antibodies are as given elsewhere herein.

The level of homodimeric HNL in the sample above a certain threshold, or a control value, is correlated with the presence of an homodimeric HNL-related disorder, while a level of homodimeric HNL in the sample below said threshold, or the control value, is indicative of low likelihood of the presence of the HNL-related disorder. Similarly, a level of homodimeric HNL above a certain threshold, a control value, is correlated with the presence of a bacterial infection, while a level homodimeric HNL below said threshold, or the control value, is indicative of low likelihood of the presence of the bacterial infection.

A threshold that differentiates most of the normal population from most of the diseased population can be determined using any well-known in the art method. Alternatively, end point values for negative (an absence of the condition), uncertain and positive (a presence of the condition) results can be determined from the data. For example, a normal range (indicative of a negative result) can be determined, the normal range including values of most of the normal population but excluding almost all those of the diseased population. Correspondingly, a range indicative of a positive result can be determined, the range including values of most of the diseased population but excluding almost all those of the normal population.

The above method for establishing a presence or an absence of a bacterial infection may further include comparing the concentration of at least one determinant, such as a peptide, protein or isoform thereof, in the sample to a statistically validated threshold for the at least one determinant. “Determinant” may be understood as a polypeptide whose amount under the disease condition is different from that under the normal condition. Exemplary determinants may include TRAIL, IL1RA, IP 10, Mac-2BP, B2M, BCA-1, CHI3L1, Eotaxin, ILla, MCP, CD62L, VEGFR2, CHP, CMPK2, COROIC, EIF2AK2, ISG15, RPL22L1, RTN3, CD112, CD134, CD182, CD231, CD235A, CD335, CD337, CD45, CD49D, CD66A/C/D/E, CD73, CD84, EGFR, GPR162, HLA-A/B/C, ITGAM, NRG1, RAP IB, SELI, SPINT2, SSEA1, IgG non-specific bound molecules, IL1, 1-TAC, TNFR1, ABTB1, ADIPOR1, ARHGDIB, ARPC2, ATP6V0B, Clorf83, CD15, CES1, COROIA, CRP, CSDA, EIF4B, EPSTII, GAS 7, HERC5, IFI6, KIAA0082, IFIT1, IFIT3, IFITM1, IFITM3, LIPT1, IL7R, ISG20, LOC26010, LY6E, LRDD, LTA4H, MAN1C1, MBOAT2, MX1, NPM1, OAS2, PARP12, PARP9, QARS, RAB13, RAB31, RAC2, RPL34, PDIA6, PTEN, RSAD2, SART3, SDCBP, TRIM 22, SMAD9, SOCS3, UBE2N, XAF1 or ZBP1. The above determinants may be used alone or in any combination.

Where available, and unless otherwise described herein, a determinant which is the product of the expression of a gene is identified based on the official letter abbreviation or gene symbol assigned by the international Human Genome Organization Naming Committee (HGNC) and listed at the US National Center for Biotechnology Information (NCBI) web site, also known as Entrez Gene.

Determinants may also encompass non-polypeptide factors, non-blood borne factors or non-analyte physiological markers of health status, such as clinical parameters as well as traditional laboratory risk factors. For example, as used herein, determinants may include non-polypeptide features (i. e. non-polypeptide determinants) such as neutrophil % (abbreviated Neu (%)), lymphocyte % (abbreviated Lym (%)), monocyte % (abbreviated Mon (%)), absolute neutrophil count (abbreviated ANC) and absolute lymphocyte count (abbreviated ALC), white blood count (abbreviated WBC), age, gender and maximal temperature (i. e. maximal core body temperature since the initial appearance of symptoms). Determinants may also include any calculated indices produced mathematically or combinations of any one of the foregoing measurements, including temporal trends and differences.

Advantageously, determinants used in the above method may include at least one determinant that is a polypeptide and at least one non-polypeptide feature.

The above method may be used for monitoring effectiveness or non-effectiveness of a therapy with an antibacterial substance, such as an antibiotic. Such therapy may be administered for treating a bacterial infection in case of e. g. sepsis. Such use may comprise repeating the above method at least twice, wherein at the first point in time the sample may be obtained prior to initiation of the therapy or part way through the therapy and at the second point in time the sample may be obtained at the completion of the therapy or part way through the therapy after the first point in time. A lesser amount of homodimeric HNL in the sample at the second point in time than that at the first point in time shall be indicative of the effectiveness of the therapy.

To increase the sensitivity of the methods disclosed herein, a pre-activator substance may be used as neutrophil activator, i.e. a substrate for activating neutrophils. The pre-activator substance may be an N-formyl peptide, preferably a tri-peptide fMLP. Advantageously, protein A may be used. Additionally, other neutrophil activators may be used. At least one of a lipopolysaccharide (LPS), a platelet-activating factor, an unmethylated CpG oligonucleotide and a tumor necrosis factor (TNF) may be used. Advantageously, fMLP may be used alone or in combination with one or more of the other neutrophil activators mentioned above.

The above methods according to the first and second aspects, respectively, may be used for distinguishing between a bacterial infection and a non-bacterial infection, such as a viral infection, between a mixed infection (i. e. a co-infection with bacteria and virus) and a viral infection. A person skilled in the art would be able to employ the method in accordance with the invention for achieving the above goals. The bacterial infection may be any one of sepsis, a gastrointestinal tract infection, a urinary tract infection, a tuberculosis, a pneumonia and a gonorrhoea.

In the methods of the present document, the sample may be first brough into contact with the second monoclonal antibody before being brought into contact with the first monoclonal antibody. The methods of the present document may comprise a step of providing a sample that is to be brought into contact with the first monoclonal antibody or the second monoclonal antibody. The methods of the present document are performed in vitro.

According to yet another aspect of the invention a kit-of-parts for detecting a presence or an absence of homodimeric HNL in a sample and/or establishing a presence or an absence of a bacterial infection, wherein the kit-of-parts comprises one or more of a monoclonal antibody as defined herein, and one or more of instructions for use, a buffer and a device, wherein the device is arranged for analysis of a sample. Preferably, said kit-of-part comprises a first monoclonal antibody and a second monoclonal antibody as defined herein. However, as the first monoclonal antibody and the second monoclonal antibody may be identical, the kit-of-parts may only comprise one antibody, which can be used as the first and second monoclonal antibody in a method of the present document.

The sample may be derived from at least one of blood, plasma, serum, urine, CSF, bone marrow, saliva, sputum, intestinal fluid, another bodily fluid, faeces and a liquid preparation thereof.

The kit-of-parts may further include an enzyme, or the second monoclonal antibody may be labelled with an enzyme for use as a signal-generating means. The kit-of-parts may further include a substrate for the enzyme and a substrate precursor that provides a detectable chromophore or fluorophore.

The above buffer of the kit-of-parts may include at least one of a block buffer and a lysis buffer.

At least one of the reagents of the kit-of-parts may be provided in a powder form, e. g. as lyophilised reagent.

At least a part of the components of kit-of-parts may be pre-attached to a solid support or may be applied to a surface of the solid support when the kit is in use.

The signal-generating means may come pre-associated with the monoclonal antibody described herein. The signal-generating means may require being combined prior to use with at least one of the buffer, an antibody-enzyme conjugate, an enzyme substrate and the like. The solid phase surface may be in a form of a tube, a bead, a microtiter plate, a microsphere or another form suitable for immobilising at least one of proteins, peptides and polypeptides. The kit-of-parts may further comprise at least one agent for detecting and measuring other biological parameters, e. g. an agent for measuring the expression and/or the amount of at least one of procalcitonin, C-reactive protein, CD64, any one of the determinants (polypeptide determinants) mentioned herein and an agent for the determination of numbers of white blood cells, such as neutrophils, T-cells, B-cells, monocytes, eosinophils and basophils.

Containers for use in the kit-of-parts may typically comprise at least one of a vial, a test tube, a flask, a bottle, a syringe and another suitable container.

The device arranged for analysis of a sample may be as described herein below.

According to yet another aspect of the invention a device is provided for establishing a presence or an absence of a bacterial infection, wherein the device comprises:
a first compartment, wherein the first compartment comprises monoclonal antibodies, a first monoclonal antibody and a second monoclonal antibody, for detecting homodimers of HNL, each of the homodimers consisting of a first monomer and a second monomer, the first monoclonal antibody being configured to bind to the first monomer, the second monoclonal antibody being configured to bind to the second monomer,
wherein each of the first monoclonal antibody and the second monoclonal antibody comprises a heavy chain variable region and a light chain variable region,
wherein the heavy chain variable region comprises a first set of murine complementarity-determining regions (CDRs), the first set of the murine CDRs comprising:
a first heavy chain CDR (CDRH1), wherein CDRH1 is SEQ ID NO: 6,
a second heavy chain CDR (CDRH2), wherein CDRH2 is SEQ ID NO: 7 and
a third heavy chain CDR (CDRH3), wherein CDRH3 is SEQ ID NO: 8,
wherein the light chain variable region comprises a second set of murine complementarity-determining regions (CDRs), the second set of the murine CDRs comprising:
a first light chain CDR (CDRL1), wherein CDRL1 SEQ ID NO: 9,
a second light chain CDR (CDRL2), wherein CDRL2 SEQ ID NO: 10 and
a third light chain CDR (CDRL3), wherein CDRL3 SEQ ID NO: 11,
wherein the first compartment is suitable for being brought into contact with a sample, the sample being suspected contain or not contain homodimers of HNL;
a second compartment, wherein the second compartment comprises a substrate for activating neutrophils present in the sample, wherein the activating occurs prior to the establishing a presence or an absence of a bacterial infection, wherein the second compartment comprises an orifice connecting it with the first compartment;
a first connector, wherein the first connector is arranged to connect the device with another device for removal of a sample from a subject suspected to have at least one of a bacterial infection and a viral infection; and
a second connector, wherein the second connector is arranged to combine the device with a computer and/or an apparatus for analysing interactions between homodimers of HNL, the first monoclonal antibody and the second monoclonal antibody, the analysing permitting determining the amount of homodimeric HNL in the sample. Details regarding the first and second monoclonal antibodies and the substrate for neutrophil activation are as disclosed elsewhere herein.

The device may be a test strip with immobilised monoclonal antibodies on at least a part of its surface, particles carrying immobilised monoclonal antibodies on at least a part of its surface. The surface may be three-dimensional, e. g. particles with a partially porous surface (layer) may carry the above monoclonal antibodies. It is important that the device comprises a contact area that can be exposed to the sample. The device may be connected to a computer or an apparatus allowing analysis and measurement of the characteristics of the interaction between the monoclonal antibodies disclosed herein and the HNL homodimers present in the sample. The interaction may be characterised using a signal that is specifically generated upon binding of a monoclonal antibody disclosed herein to a homodimer of HNL (no such signal is generated in a control set-up, wherein no above interaction occurs). The signal can be at least one of a physical signal, a chemical signal and a biological signal. Examples of the signal include a chromogenic signal, a fluorogenic signal, a spectroscopically measurable signal, a change in ionization, a change in conductivity and the like.

The device may further comprise a binding agent or an antibody for detecting one or more of the above determinants. The device may involve magnetic separation of the analytes of interest or the like prior to their detection.

The device may be any one of a point-of-care device, a test strip, a device for a radioimmunoassay, a device ELISA, a device for a “sandwich” immunoassay, a device for a immunoradiometric assay, a device for a gel diffusion precipitation assay, a device for a immunodiffusion assay, a device for an in situ immunoassay using colloidal gold, enzyme or radioisotope labels, a device for a Western blot, a device for a precipitation assay, a device for a gel agglutination assay, a device for a hemagglutination assay, a device for a immunofluorescence assay, a device for a protein A assay, a device for a immunoelectrophoresis assay and a device for a detection of analytes that involve magnetic separation of the analytes.

It is appreciated that the above-mentioned embodiments of the first aspect of the present invention may also apply, even if not explicitly mentioned, to the further aspects of the present invention.

The invention has mainly been described above with reference to some embodiments. However, as it is readily appreciated by the person skilled in the art, other embodiments than the ones disclosed above are equally possible within the scope of the invention as defined by the appended claims.

### EXPERIMENTAL PROCEDURES

The present examples are provided for illustrative purposes only and should not be construed to be limiting the invention as defined by the appended claims. All references provided below and elsewhere in the present application are hereby included herein via reference.

### Production of Antibodies

Monoclonal antibodies Anti-HNL MAB2 and Anti-HNL MAB3 were produced by the standard hybridoma technique by the fusion of spleen cells from HNL dimer immunised Balb/c mice with Sp2/0 mouse myeloma cells, followed by cloning. Supernatants were screened for activity against purified HNL with help of ELISA. Selected hybridomas were expanded and antibodies purified from these were of the IgGI subtype.

### Characterisation of Antibodies

Using chemical cross-linking, high-mass MALDI mass spectrometry and nLC-Orbitrap mass spectrometry the interaction interface between the antigen HNL and each of the monoclonal antibody Anti-HNL MAB2 and Anti-HNL MAB3 was characterised (see WO 2016/079219 A1).

The epitope of each of Anti-HNL MAB2 and Anti-HNL MAB3 includes amino acids 145-154 as shown in SEQ ID No: 26 of WO 2016/079219. Additionally, the epitope of Anti-HNL MAB2 includes amino acids 83-88 as shown in SEQ ID No: 26 of WO 2016/079219.

### Proof-of-Concept

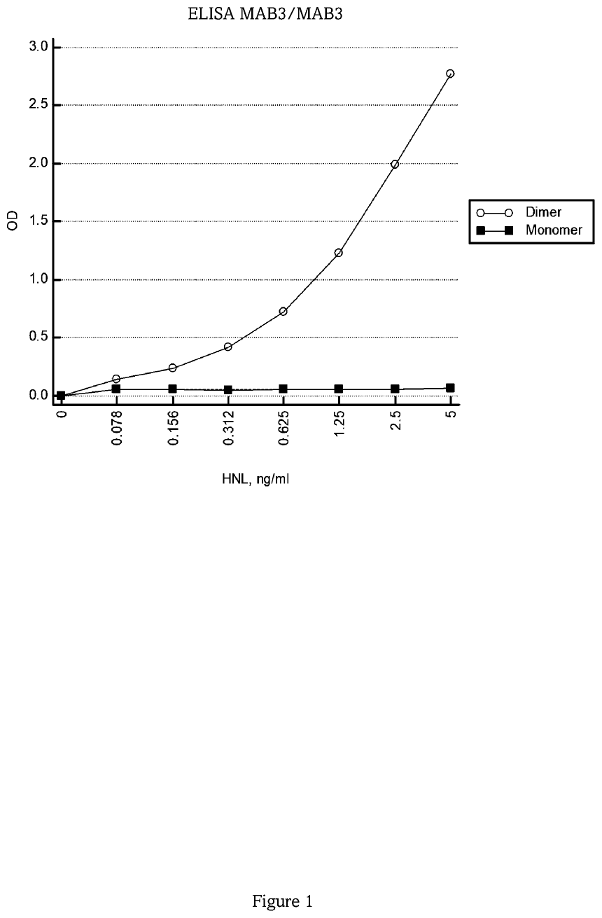

Performing the method for detecting a presence or an absence of homodimeric human neutrophil lipocalin (HNL) in a sample of the present document using ELISA with the first monoclonal antibody MAB3 and the second monoclonal antibody MAB3 specific binding to and detection of homodimers of HNL was achieved (FIG. 1).

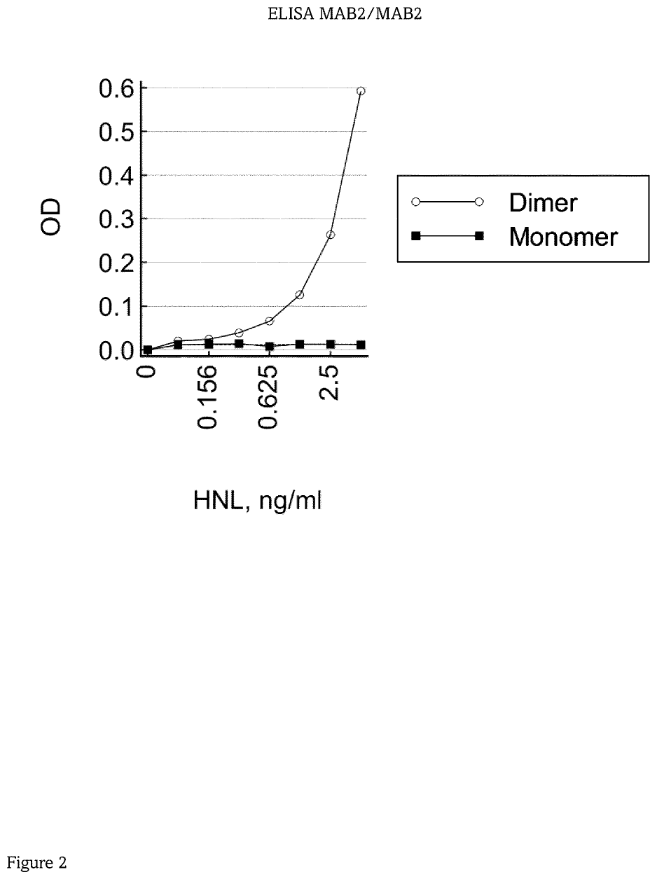

Performing the method of the present document using ELISA with the first monoclonal antibody MAB2 and the second monoclonal antibody MAB2 specific binding to and detection of homodimers of HNL was achieved (FIG. 2).

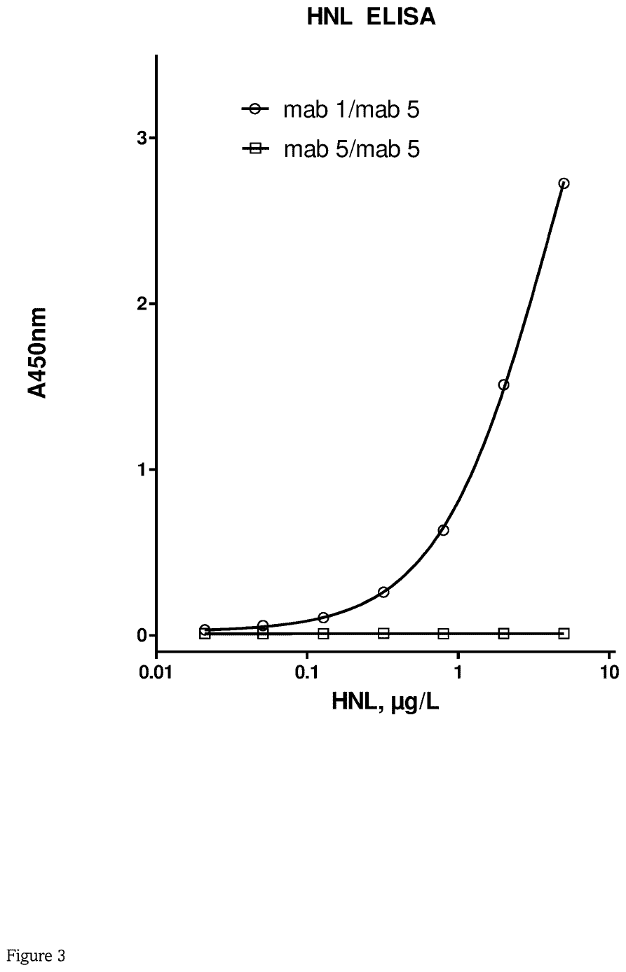

For comparison, FIG. 3 shows the results of performing the method of the present document using a first and second monoclonal antibody (MAB5) not having the CDR sequences of the first and second monoclonal antibodies as defined herein. As can be seen in FIG. 3, using MAB5 as both first monoclonal antibody and the second monoclonal antibody dimeric HNL cannot be detected. However, when MAB5 is used together with another anti-HNL monoclonal antibody (MAB1) dimeric HNL can be detected (thus demonstrating that MAB5 is able to bind to HNL in dimeric form). Thus, not any combination of monoclonal antibodies specific for HNL, wherein the first and second monoclonal antibodies have the same CDR sequences, may be used in a method according to the present document for detecting a presence or an absence of homodimeric human neutrophil lipocalin (HNL) in a sample.

### Analysis of Clinical Samples Using Anti-HNL MAB3

The patient group consisted of 206 patients, each of which had signs of infection and body temperature of more than 38 °C. The aetiologies of the infections were determined by routine clinical procedures. No patient took immunosuppressive medication. For each patient, the infection was either bacterial or a viral infection. The bacterial infections included in the study were bacterial pneumonia, a mycoplasma infection, tonsillitis, a urinary tract infection (UTI), a gastrointestinal (GI) infection, erysipelas and sepsis. In the majority of cases the bacterial infection was an acute respiratory infection, such as pneumonia and tonsillitis.

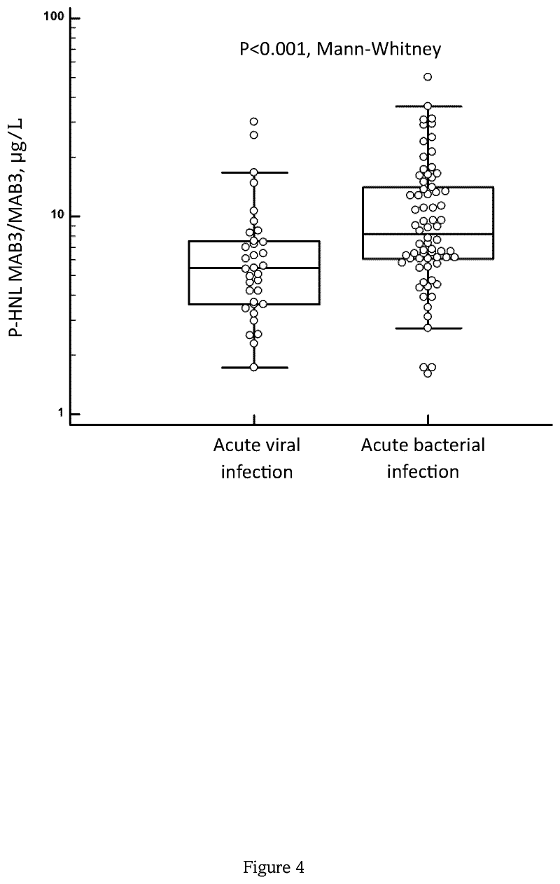

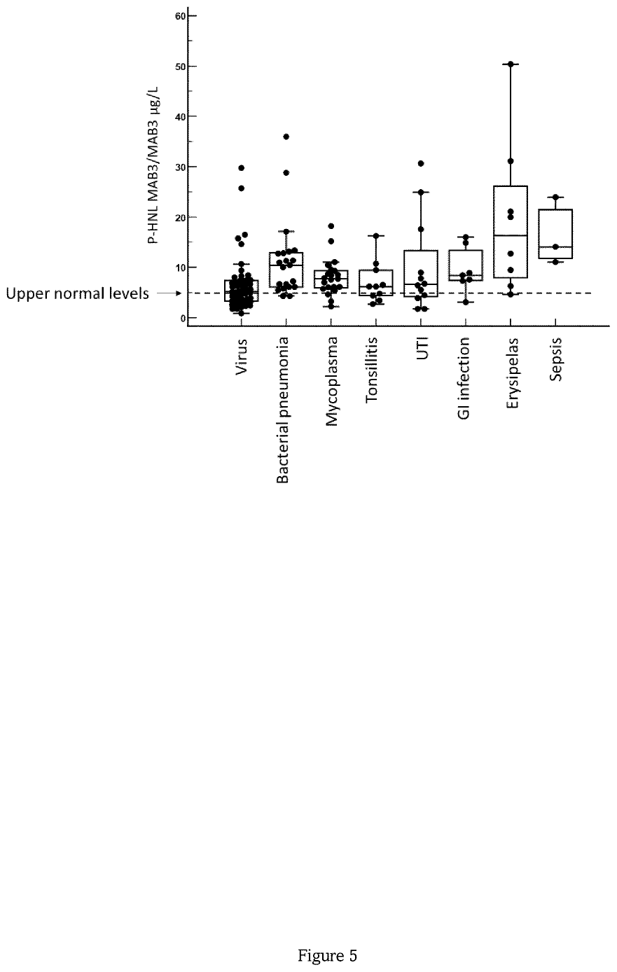

The blood samples were drawn before initiation of antibacterial treatment, anticoagulated with EDTA and subjected to centrifugation at 3000 g. Following the centrifugation of the blood samples, the plasma was harvested and stored at −70 °C before analysis. Using ELISA as described herein, concentration of HNL MAB3/MAB3 (the immunocomplexes between homodimeric HNL and the monoclonal antibodies) in plasma was determined. It was demonstrated that using the above method it was possible to reliably and accurately detect both the presence of an acute bacterial infection and the absence thereof (e. g. an acute viral infection only, no bacterial infection) (FIGS. 4 and 5).

1. An in vitro method for detecting a presence or an absence of homodimeric human neutrophil lipocalin (HNL) in a sample, wherein the method comprises:
providing a sample,
providing monoclonal antibodies, a first monoclonal antibody and a second monoclonal antibody, for detecting homodimers of HNL, each of the homodimers consisting of a first monomer and a second monomer, the first monoclonal antibody being configured to bind to the first monomer, the second monoclonal antibody being configured to bind to the second monomer,
bringing the sample into contact with the first monoclonal antibody,
bringing the sample into contact with the second monoclonal antibody,
detecting a presence or an absence of homodimeric HNL,
wherein each of the first monoclonal antibody and the second monoclonal antibody comprises a heavy chain variable region and a light chain variable region,
wherein the heavy chain variable region comprises a first set of murine complementarity-determining regions (CDRs), the first set of the murine CDRs comprising:
a first heavy chain CDR (CDRH1), wherein CDRH1 is SEQ ID NO: 6,
a second heavy chain CDR (CDRH2), wherein CDRH2 is SEQ ID NO: 7 and
a third heavy chain CDR (CDRH3), wherein CDRH3 is SEQ ID NO: 8,
wherein the light chain variable region comprises a second set of murine complementarity-determining regions (CDRs), the second set of the murine CDRs comprising:
a first light chain CDR (CDRL1), wherein CDRL1 SEQ ID NO: 9,
a second light chain CDR (CDRL2), wherein CDRL2 SEQ ID NO: 10 and
a third light chain CDR (CDRL3), wherein CDRL3 SEQ ID NO: 11.

2. The in vitro method according to claim 1, wherein the sample is derived from at least one of blood, plasma, serum, urine, CSF, bone marrow, saliva, sputum, intestinal fluid, another bodily fluid, faeces and/or a liquid preparation thereof.

3. The in vitro method according to claim 1, wherein the first monoclonal antibody is immobilised on a solid support.

4. The in vitro method according to claim 1, wherein each of the CDRs are configured to bind to an epitope formed between amino acids 145 to 154 of HNL as defined in SEQ ID NO: 1.

5. The in vitro method according to claim 1, wherein at least one of the first monoclonal antibody and the second monoclonal antibody has a heavy chain comprising the first set of the murine CDRs, wherein the heavy chain comprises a sequence as depicted in SEQ ID NO: 5 or an amino acid sequence having at least 80%, of identity to SEQ ID NO: 5.

6. The in vitro method according to claim 1, wherein at least one of the first monoclonal antibody and the second monoclonal antibody has a light chain comprising the second set of the murine CDRs, wherein the light chain comprises a sequence as depicted in SEQ ID NO: 4 or an amino acid sequence having at least 80%, of identity to SEQ ID NO: 4.

7. The in vitro method according to claim 1, wherein at least one of the first monoclonal antibody and the second monoclonal antibody has a heavy chain comprising the first set of the murine CDRs, wherein the heavy chain comprises a sequence as depicted in SEQ ID NO: 3 or an amino acid sequence having at least 80%, of identity to SEQ ID NO: 3.

8. The in vitro method according to claim 1, wherein at least one of the first monoclonal antibody and the second monoclonal antibody has a light chain comprising the second set of the murine CDRs, wherein the light chain comprises a sequence as depicted in SEQ ID NO: 2 or an amino acid sequence having at least 80%, of identity to SEQ ID NO:2.

9. The in vitro method according to claim 1, wherein each of the first monoclonal antibody and the second monoclonal antibody has a heavy chain comprising the first set of the murine CDRs, wherein the heavy chain comprises a heavy chain variable region sequence as depicted in SEQ ID NO: 5 or an amino acid sequence having at least 80%, of identity to SEQ ID NO: 5, and wherein each of the first monoclonal antibody and the second monoclonal antibody has a light chain comprising the second set of the murine CDRs, wherein the light chain comprises a light chain variable region sequence as depicted in SEQ ID NO: 4 or an amino acid sequence having at least 80% of identity to SEQ ID NO: 4.

10. The in vitro method according to claim 1, wherein each of the first monoclonal antibody and the second monoclonal antibody has a heavy chain comprising the first set of the murine CDRs, wherein the heavy chain comprises a heavy chain variable region sequence as depicted in SEQ ID NO: 3 or an amino acid sequence having at least 80% of identity to SEQ ID NO: 3, and wherein each of the first monoclonal antibody and the second monoclonal antibody has a light chain comprising the second set of the murine CDRs, wherein the light chain comprises a light chain variable region sequence as depicted in SEQ ID NO: 2 or an amino acid sequence having at least 80% of identity to SEQ ID NO: 2.

11. The in vitro method according to claim 1, wherein at least one of the first monoclonal antibody and the second monoclonal antibody is a humanised antibody.

12. An in vitro method for establishing a presence or an absence of a bacterial infection, the method comprising:
providing a sample,
providing monoclonal antibodies, a first monoclonal antibody and a second monoclonal antibody, for detecting homodimers of HNL, each of the homodimers consisting of a first monomer and a second monomer, the first monoclonal antibody being configured to bind to the first monomer, the second monoclonal antibody being configured to bind to the second monomer,
bringing the sample into contact with the first monoclonal antibody,
bringing the sample into contact with the second monoclonal antibody,
determining a value corresponding to the amount of homodimeric HNL in the sample, comparing the value with a control value,
establishing a presence of a bacterial infection when the value is significantly higher than the control value or establishing an absence of a bacterial infection when the value is significantly lower than the control value,
wherein said first monoclonal antibody and/or second monoclonal antibody is as defined in claim 1.

13. A kit-of-parts for detecting a presence or an absence of homodimeric HNL in a sample according to claim 1 and one or more of instructions for use, a buffer and a device, wherein the device is arranged for analysis of a sample.

14. A device for establishing a presence or an absence of a bacterial infection, the device comprising:
a first compartment, wherein the first compartment comprises monoclonal antibodies, a first monoclonal antibody and a second monoclonal antibody, for detecting homodimers of HNL, each of the homodimers consisting of a first monomer and a second monomer, the first monoclonal antibody being configured to bind to the first monomer, the second monoclonal antibody being configured to bind to the second monomer,
wherein said first monoclonal antibody and/or second monoclonal antibody is as defined in claim 1,
wherein the first compartment is suitable for being brought into contact with a sample, the sample being suspected to contain or not contain homodimers of HNL;
a second compartment, wherein the second compartment comprises a substrate for activating neutrophils present in the sample, wherein the activating occurs prior to the establishing a presence or an absence of a bacterial infection, wherein the second compartment comprises an orifice connecting it with the first compartment;
a first connector, wherein the first connector is arranged to connect the device with another device for removal of a sample from a subject suspected to have at least one of a bacterial infection and a viral infection; and
a second connector, wherein the second connector is arranged to combine the device with a computer and/or an apparatus for analysing interactions between homodimers of HNL, the first monoclonal antibody and the second monoclonal antibody, the analysing permitting determining the amount of homodimeric HNL in the sample.

15. The device according to claim 14, the device further comprising a binding agent for one or more determinants selected from the group consisting of ABTB1, ADIPOR1, ARHGDIB, ARPC2, ATP6V0B, Clorf83, CD15, CES1, COROIA, CSDA, EIF4B, EPSTII, GAS 7, HERC5, IFI6, KIAA0082, IFIT1, IFIT3, IFITM1, IFITM3, LIPT1, IL7R, ISG20, LOC26010, LY6E, LRDD, LTA4H, MANICI, MBOAT2, NPM1, OAS2, PARP12, PARP9, QARS, RAB13, RAB31, RAC2, RPL34, PDIA6, PTEN, RSAD2, SART3, SDCBP, SMAD9, SOCS3, TRIM22, SART3, UBE2N, XAF1, ZBP1, CRP and MX1.

16. The device according to claim 14, wherein the device is any one of a point-of-care device, a test strip, a device for a radioimmunoassay, a device ELISA, a device for a “sandwich” immunoassay, a device for a immunoradiometric assay, a device for a gel diffusion precipitation assay, a device for a immunodiffusion assay, a device for an in situ immunoassay using colloidal gold, enzyme or radioisotope labels, a device for a Western blot, a device for a precipitation assay, a device for a gel agglutination assay, a device for a hemagglutination assay, a device for a immunofluorescence assay, a device for a protein A assay, a device for a immunoelectrophoresis assay and a device for a detection of analytes that involve magnetic separation of the analytes.


SEQUENCE LISTING (11 sequences)

SEQ ID NO: 1 - protein, 198 aa, Homo sapiens
MPLGLLWLGLALLGALHAQAQDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 2 - protein, 115 aa, Mus musculus
DIVLTQSTSSLSVSLGDRVTINCRASQDISNYLNWYQEKPDGTVKLLIYFTSRLHSGVPSRFSGSGSGTDYSLTITNLEQEDIATYFCQQGNTLPWTFGGGTKLEIKRADAAPTV

SEQ ID NO: 3 - protein, 118 aa, Mus musculus
EVQLEESGPGLVAPSQSLSITCTISGFSLTSYGIHWLRQPPGKDLEWLVVIWGDGSTTSNSALKSRLSISKDNSKSQVFFKMSGLQTDDTAIYYCARHRYSDYHAMDYWGPGTSVTVS

SEQ ID NO: 4 - protein, 115 aa, Mus musculus
DIVLTQTTSSLSVSLGDRVTINCRASQDISNYLNWYQEKPDGTVKLLIYFTSRLHSGVPSRFSGSGSGTDYSLTITNLEQEDIATYFCQQGNTLPWTFGGGTKLEIKRADAAPTV

SEQ ID NO: 5 - protein, 119 aa, Mus musculus
EVKLQESGPGLVAPSQSLSITCTISGFSLTSYGIHWLRQPPGKDLEWLVVIWGDGSTTSNSALKSRLSISKDNSKSQVFFKMSGLQTDDTAIYYCARHRYSDYHAMDYWGPGTSVTVSS

SEQ ID NO: 6 - protein, 10 aa, Mus musculus
GFSLTSYGIH

SEQ ID NO: 7 - protein, 16 aa, Mus musculus
VIWGDGSTTSNSALKS

SEQ ID NO: 8 - protein, 11 aa, Mus musculus
HRYSDYHAMDY

SEQ ID NO: 9 - protein, 11 aa, Mus musculus
RASQDISNYLN

SEQ ID NO: 10 - protein, 7 aa, Mus musculus
FTSRLHS

SEQ ID NO: 11 - protein, 9 aa, Mus musculus
QQGNTLPWT